(19) United States
(12) Patent Application Publication
(10) Pub. No.: US 2017/0101437 A1
(43) Pub. Date: Apr. 13, 2017

(54) PROCESS FOR PURIFICATION OF DARBEPOETIN ALFA

(71) Applicant: Hetero Drugs Ltd., Telengana (IN)

(72) Inventors: Vamsi Krishna Bandi, Hydrabad (IN); Bala Reddy Bheemareddy, Hydrabad (IN); Shivu Mokshanath Mugthihalli, Hydrabad (IN); Pradeep Kumar Iyer, Hydrabad (IN); Prasad Pasupuleti, Hydrabad (IN)

(21) Appl. No.: 15/046,143

(22) Filed: Feb. 17, 2016

(30) Foreign Application Priority Data
Oct. 9, 2015 (IN) 5410/CHE/2015

(51) Int. Cl. C07K 1/36; C07K 14/505; C07K 1/22; C07K 1/16; C07K 1/18

(52) U.S. Cl. CPC C07K 1/36; C07K 1/165; C07K 1/18; C07K 1/22; C07K 14/505

(57) ABSTRACT

The present invention provides sequential chromatography steps and conditions for purification of a recombinant protein, especially recombinant darbepoetin alfa.

### RELATED APPLICATION

This application is filed as a Complete Application with the Indian Patent Office.

### FIELD OF INVENTION

The invention provides sequential steps and conditions for purification of protein, especially recombinant darbepoetin alfa.

### BACKGROUND

Purification of recombinant proteins is key to having an effective drug product. Erythropoietin and its analogues have sugar moieties that make up the glycan chains. These essentially contribute to the heterogeneity in the isoforms of these proteins, thus challenging the purification process. The sialic acid content in both erythropoietin and its analogue darbepoetin alpha determines the overall charge and isoelectric point (See Rush et al;, Analytical Chemistry 1995, 67:1442). It is also widely reported that low pl isoforms exhibit higher bioactivity than those with high pl isoforms (See Zanette et al., Journal of Biotechnology 2003, 101:275). In darbepoetin alpha, there is a heterogeneous mixture of isoforms with pl ranging from 3 to 8 although the drug Aranesp (darbepoetin alpha) comprises low isoforms having a pl range of 3 to 3.9. Thus there is a need for better purification methods to address the heterogeneous mixtures of isoforms present in these protein.

Earlier method of erythropoietin purification described in WO198607594 has involved reverse phase liquid chromatographic separation using immobilized C4 or C6 resin followed by elution of bound protein from the resin with aqueous ethanol solution at a pH of about 5 to 8. WO2003045996 discloses purification of recombinant human erythropoietin by reverse phase chromatography followed by anion exchange chromatography and size exclusion chromatography.

WO2011063195 discloses a process to purify low pl isoforms of darbepoetin alpha from higher pl isoforms comprising at least one cation exchange chromatographic (CEX) step in bind-elute mode and one CEX step in flow-through mode. The above process allows for other intermediary anion exchange or mixed mode chromatographic steps before or after the two CEX steps.

All of the above processes however involve complex steps which may not achieve desired percentage purity of the drug product. The present invention provides sequential chromatographic and filtrations steps for purification of erythropoietin and its analogues, especially darbepoetin alfa resulting in 98% pure drug substance.

### BRIEF DESCRIPTION OF DRAWINGS

FIG. 1 shows the results of Isoelectric Focusing (IEF) analysis for Isoform distribution. Lanes 1 and 8: 1 μg Standard (system suitability); Lanes 2 and 9: 5 μg Standard; Lane 3: Anion Exchange Chromatography Input—Bag 1; Lane 4: Anion Exchange Chromatography Input—Bag 2; Lane 5: Anion Exchange Chromatography I Eluate Cycle 1; Lane 6: Anion Exchange Chromatography I Eluate Cycle 2; Lane 7: Anion Exchange Chromatography II Eluate; Lane 10: Anion Exchange Chromatography II Eluate; Lane 11: Cation Exchange Chromatography—Flow through; Lane 12: Anion Exchange Chromatography III Eluate; Lane 13: Gel filtration chromatography—pooled;Lane 14: Drug Substance.

FIG. 2 shows SDS-PAGE analysis for molecular weight related substances. Lanes 1 and 9: Marker; Lanes 2 and 10: Standard; Lanes 3 and 11: 1% Standard; Lane 4: Anion Exchange Chromatography Input—Bag 1; Lane 5: Anion Exchange Chromatography Input—Bag 2; Lane 6: Anion Exchange Chromatography I Eluate Cycle 1; Lane 7: Anion Exchange Chromatography I Eluate Cycle 2; Lane 8: Anion Exchange Chromatography II Eluate; Lane 12: Anion Exchange Chromatography II Eluate; Lane 13: Anion Exchange Chromatography III Eluate; Lane 14: Gel filtration Chromatography—pooled; Lane 15: Drug Substance.

FIG. 3 shows the analysis of product purity by Size Exclusion Chromatography.

A3-E/GFC-IP: Anion Exchange Chromatography III eluate/Gel Filtration chromatography—Input; GFC-OP: Gel filtration Chromatography—Output.

### SUMMARY

The present invention provides a purification process for the purification of recombinant protein, especially the recombinant erythropoietin and its analogue darbepoetin alfa. In one aspect, the process of the invention comprises multiple sequential steps of chromatography:
(a) Anion Exchange Chromatography I;
(b) Mixed Mode Chromatography;
(c) Anion Exchange Chromatography II;
(d) Cation Exchange Chromatography;
(e) Affinity Chromatography;
(f) Anion Exchange Chromatography III;
(g) Gel Filtration chromatography.

In another aspect, the process consists of chromatographic steps interspersed with Buffer Exchanges.

The invention, in another key aspect, provides low pH treatment at 3.7 for 1 hour after the step of Mixed Mode Chromatography.

In yet another aspect, buffer exchange is carried out at least in three stages:
(a) After Anion Exchange Chromatography I;
(b) After Anion Exchange Chromatography II;
(c) After Affinity Chromatography.

The process of purification disclosed herein results in protein which is about 98.0 to 99.8% pure.

### DETAILED DESCRIPTION

The recombinant protein, for example, darbepoetin alfa is produced in Chinese Hamster Ovary (CHO) cells using appropriate culture conditions, temperature and pH. The present invention provides an efficient purification of recombinant proteins. In one embodiment, the purification process is provided especially for recombinant erythropoietin and its analogues, especially darbepoetin alfa. The process of purification consists of multiple chromatography steps interspersed with filtration steps.

The process of purification of protein of the invention comprises the following steps:
(a) passing clarified filtered harvest through anion exchange chromatography I yielding eluate;
(b) eluate of (a) is subjected to buffer exchange I;
(c) process of buffer exchange I is followed by loading onto mixed mode chromatography (MMC);
(d) MMC of (c) is followed by low pH treatment at pH 3.7 and readjustment to pH 4.5;
(e) step (d) is followed sequentially by anion exchange chromatography II and buffer exchange II;
(f) eluate of step (e) is loaded onto cation exchange chromatography;
(g) eluate of step (f) is loaded onto affinity chromatography step;
(h) affinity eluate of step (g) is subjected to buffer exchange III followed by anion exchange chromatography III;
(i) aggregates in the eluate from step (h) is filtered by gel filtration chromatography;
(j) process of (i) is followed by nano filtration to remove viruses.

The diluted drug product obtained from the above process is about 98.0 to 99.8% pure. In a preferred embodiment, recombinant darbepoetin alfa purified by the process of the present invention is 99.8% pure.

In detail, the purification process of the present invention involves sequential steps as described below:

(A) Anion Exchange Chromatography I: This step, which is the initial step in the purification process is carried out with the clarified, filtered harvest resulting from the upstream process. In one embodiment, anion exchange chromatography I consists of Tris and NaCl at a pH of 7.2 which serves as equilibration and wash I buffer. Wash Buffer II consists of sodium acetate at pH 6.0. Wash buffer III consists of increased strength of sodium acetate at a pH of 3.7. The protein is further washed with wash buffer IV which also serves as pre-elution buffer using Tris at pH 7.2. Elution buffer consists of Tris and 200 mM NaCl at a pH of 7.2. The protein is further subjected to regeneration buffer consisting of Tris and 1M NaCl at pH 7.2.

In a preferred embodiment, the anion exchange chromatographyl (AEX-I) of the purification process is for the removal of almost 80% impurities. This step also facilitates lower column volumes for the following chromatography steps in addition to reducing the buffer volumes for the remaining steps of purification. Thus, AEX-I serves as a step for cost reduction in the downstream process.

It is also provided that 65% of the basic impurities are removed in flow through during AEX-I; wash III removes up to 10% of impurities; regeneration removes approximately 5% of impurities. Eluate following AEX, washings and regeneration leads to about collection of 20% of the loaded protein.

It is envisaged that AEX-I step removes basic impurities, HCP, DNA, endotoxins and viruses. Buffer exchange-I is carried out for loading the AEX-I eluate on to the next step of mixed mode chromatography. The equilibration buffer used in this step is sodium acetate and sodium phosphate buffer at a pH of 6.0.

(B) Mixed Mode Chromatography (MMC): Mixed mode chromatography step in the purification process is performed to remove basic isoform impurities, HCP and DNA. This step involves use of equilibration and wash I buffer consisting of sodium acetate and sodium phosphate at pH 6.0 followed by regeneration consisting of regeneration buffer of 500 mM sodium phosphate at pH 6.8.

In yet another embodiment, as a key step towards virus inactivation, low pH treatment is carried out at pH 3.7 for 1 hr. This step inactivates the enveloped viruses (≧4 Log₁₀Reduction Value, LRV). Tris is used to readjust the pH to 4.5.

(C) Anion Exchange Chromatography II: For further concentration of the protein and removal of HCP, DNA and all types of viruses (≧4 LRV), AEX-II is carried out. Further buffer exchange-II serves as an intermediary step between AEX-II eluate and loading of the protein onto cation exchange chromatography (CEX).

(D) Cation Exchange Chromatography (CEX):In a preferred embodiment, the CEX is performed to control the isoform profile thus rendering the standard product quality. This step also removes HCP from the protein sample. The invention particularly provides the loading condition, such as pH, allowing higher resolution of isoforms as compared to routine cation exchange resins.

(E) Affinity Chromatography: In yet another embodiment affinity chromatography step is carried out in the presence of equilibration buffer and elution buffer, each with different concentrations of NaCI to remove degraded protein that is generated in the CEX. Affinity eluate is conditioned with buffer exchange-Ill in order to load on to anion exchange chromatography III step.

(F) Anion Exchange Chromatography Ill (AEX-III): The invention provides yet another step of AEX which is primarily for concentration of the protein to be used in subsequent steps. Again, this is carried out in equilibration buffer (pH 6) and elution buffer (7.2).

(G) Gel Filtration Chromatography: In a preferred embodiment, gel filtration is used to remove aggregates. This step also provides for the protein product to be formulated in formulation buffer.

(H) Nano Filtration: This step is provided to remove all types of viruses (LI LRV) to satisfy regulatory requirement. Filtration after dilution results in drug substance which is put through bulk batch release tests and stability.

The protein of the invention purified using the above sequential steps results in ≧98% purity. In a preferred embodiment, the protein purity achieved by this process in >99%. In a still preferred embodiment, the protein purity achieved by the said process is 99.8%.

It is provided that the purification process of the invention is used for obtaining high purity drug product of recombinant proteins, especially recombinant proteins with higher sialic content. The invention provides especially for purification of recombinant erythropoietin and darbepoetin alfa isoforms. More specifically, the invention provides for purification of recombinant darbepoetin alfa.

Examples given below further aid in the understanding of the invention. However, no aspect of the example should be construed as limiting the scope of the invention.

### Method

### Upstream Process of Obtaining Darbepoetin Alfa Protein:

One vial of Chinese Hamster Ovary (CHO) cells having Darbepoetin gene construct, producing Darbepoetin was thawed from liquid nitrogen transferred to shake flask culture with 40 mL of culture medium and incubated at 37° C., 5% CO₂ with 120 RPM agitation. After 3-4 days of incubation when the cell count reached to about 2-4 million cells/mL the cells were transferred and cultured in bigger flasks in shaker incubator at 37° C., 5% CO₂ and 120 RPM. Subsequently, when the cell count reached 3-4 million cells/mL, these cells were transferred to seed bioreactor with a culture volume of 2 L in a 6.5 L glass bioreactor with an initial seeding density of 0.25 million cells/mL. The culture was agitated for 3-4 days till the cell count reached to about 2-4 million cells/mL and the process was shifted to production bioreactor with a seed density of 0.25 million cells/mL in 16 L culture medium. Cells were grown in production bioreactor for 12 days with feeding on every alternate day starting from 3^{rd} day under set parameters of temperature at 37° C., dissolved oxygen at 50% and pH at 7.0±0.1 at varying agitation speeds depending upon oxygen demand. Culture medium with darbepoetin protein was harvested on Day 12 and clarified using 0.2 μm hollow fiber to remove the cell debris.

### EXAMPLE I

Darbepoetin harvested from the upstream process was subjected to the following process of purification

A) Anion Exchange Chromatography I (AEX-I)

TABLE 1
Buffer conditions for AEX-1

| Buffer | Concentration | pH |
|---|---|---|
| Equilibration/Wash I buffer | 25 mM Tris, 60 mM NaCl | 7.2 |
| Wash II buffer | 21 mM sodium acetate | 6.0 |
| Wash III buffer | 217 mM sodium acetate | 3.7 |
| Pre-elution/Wash IV buffer | 25 mM Tris | 7.2 |
| Elution Buffer | 25 mM Tris, 200 mM NaCl | 7.2 |
| Regeneration Buffer | 25 mM Tris, 1M NaCl | 7.2 |

Buffer exchange I was performed for AEX-I eluate to load on to the next step of mixed mode chromatography. The equilibration buffer used in this step is 21 mM sodium acetate and 5 mM sodium phosphate buffer at a pH of 6.0.

B) Mixed Mode Chromatography was carried out with the following conditions given in Table 2.

TABLE 2
Buffer conditions for Mixed Mode Chromatography (MMC)

| Buffer | Concentration | pH |
|---|---|---|
| Equilibration/Wash I buffer | 21 mM sodium acetate, | 6.0 |
| | 5 mM sodium phosphate | |
| Regeneration Buffer | 500 mM sodium phosphate | 6.8 |

MMC accounted for removal of approximately 20% impurities.

Low pH treatment at pH 3.7 for 1 hr was then carried out to inactivate the enveloped viruses. Tris at 1M was used to readjust pH to 4.5.

(C) Anion Exchange Chromatography II: Conditions for AEX-II is provided in Table 3.

TABLE 3
Buffer Conditions for AEX-II

| Buffer | Concentration | pH |
|---|---|---|
| Equilibration Buffer | 51 mM sodium acetate | 4.5 |
| Elution Buffer | 25 mM Tris, 200 mM NaCl | 7.2 |
| Regeneration Buffer | 25 mM Tris, 1M NaCl | 7.2 |

Buffer Exchange II was carried out with 51mM sodium acetate at pH 4.5.

(D) Cation Exchange Chromatography (CEX): To obtain protein purity, CEX was performed.

TABLE 4
Conditions for CEX

| Buffer | Concentration | pH |
|---|---|---|
| Equilibration Buffer | 51 mM sodium acetate | 4.5 |
| Regeneration Buffer | 25 mM Tris, 1M NaCl | 7.2 |

It was found that the conditions provided resulted in higher resolution of isoforms thus resulting in highly pure protein.

(E) Affinity Chromatography: This step was carried out with the following conditions (Table 5) in order to remove the degraded protein generated in CEX.

TABLE 5

| Buffer | Concentration | pH |
|---|---|---|
| Equilibration Buffer | 21 mM sodium acetate, 10 mM NaCl | 6.0 |
| Elution Buffer | 21 mM sodium acetate, 1M NaCl | 6.0 |

Buffer exchange III with 21mM sodium acetate at pH 6.0 was carried out.

(F) Anion Exchange Chromatography III (AEX-III): Affinity chromatography was followed up with AEX-III in order to concentrate the protein prior to gel filtration. Conditions for AEX-III is provided below

TABLE 6

| Buffer | Concentration | pH |
|---|---|---|
| Equilibration Buffer | 21 mM sodium acetate | 6.0 |
| Elution Buffer | 25 mM Tris, 200 mM NaCl | 7.2 |

(G) Gel Filtration Chromatography: This step was carried out in formulation buffer at pH 6.2 resulting in removal of aggregates.

(H) Nano Filtration: As a final step before dilution and preparation of drug substance, nano filtration was carried out using 0.2 μm filtration followed by 20 nm filtration.

### EXAMPLE 2

### Protein Purity Analysis

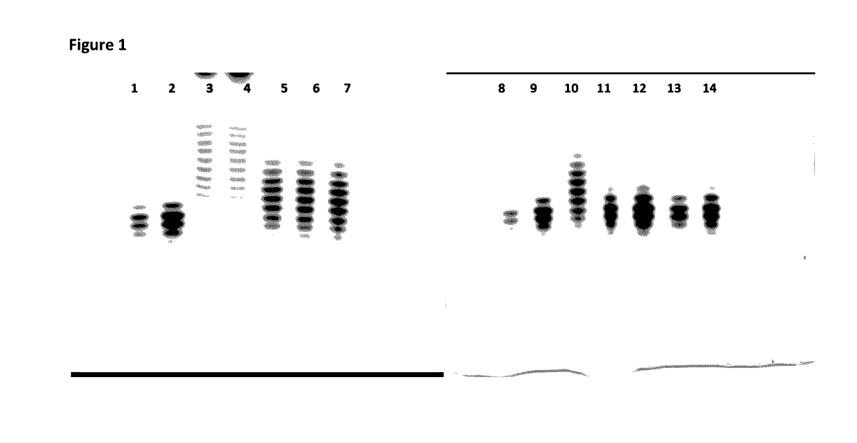

Darbepoetin alpha purified as per the steps of example 1 was analyzed using isoelectric focusing for isoform distribution. The results clearly indicates removal of unrelated glycoforms across the various process steps (FIG. 1). The final polishing is seen at the CEX where the isoform pattern is similar to that of innovator.

### EXAMPLE 3

### Analysis for Molecular Weight Related Substances

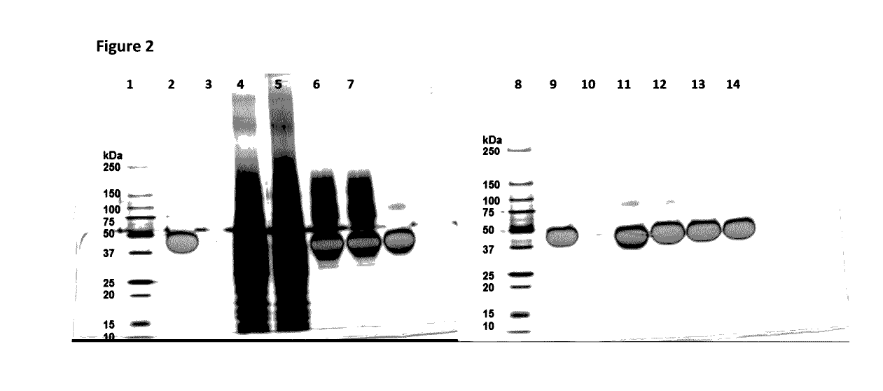

SDS-PAGE was carried out to analyze the molecular weight of the eluates from different process steps (FIG. 2). The results show reduction in low molecular weight impurities and high molecular weight impurities across the various process steps. Furthermore, removal of aggregates after the gel filtration chromatography step was seen with a purity of >99% and aggregation values less than 1%.

### EXAMPLE 4

### Analysis of Product Purity by Size Exclusion Chromatography (SEC)

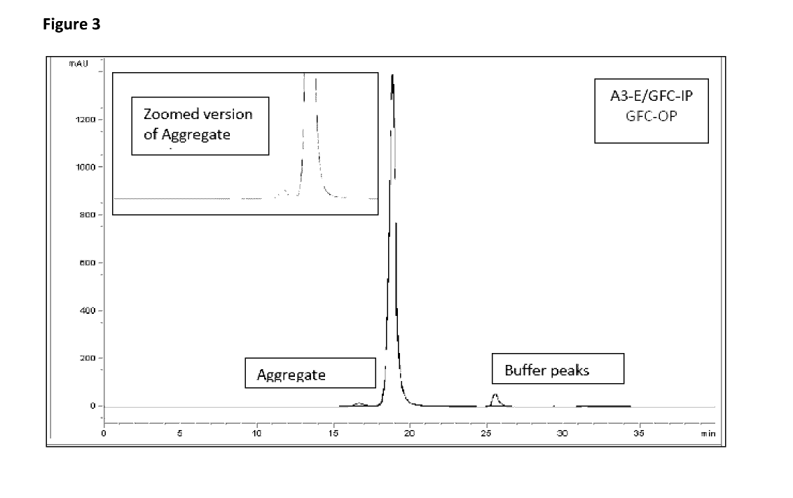

FIG. 3 shows anion exchange chromatography Ill eluate and Gel Filtration chromatography (A3-E/GFC-IP) input and Table 7 below provides the gel filtration chromatography output (GFC-OP).

TABLE 7

| Sample | % HMW Impurities | % Purity |
|---|---|---|
| A3-E/GFC-IP | 1.4 | 98.6 |
| GFC-OP | 0.2 | 99.8 |

Above results indicate quantitative reduction of aggregates in the GFC step as demonstrated by SEC.

We claim:

1. A process of purification of a recombinant protein, the said process comprising the sequential chromatographic steps of:
(a) Anion Exchange Chromatography I;
(b) Mixed Mode Chromatography;
(c) Anion Exchange Chromatography II;
(d) Cation Exchange Chromatography;
(e) Affinity Chromatography;
(f) Anion Exchange Chromatography III;
(g) Gel filtration chromatography.

2. The process of claim 1, wherein
(a) Anion Exchange Chromatography I is followed by Buffer exchange;
(b) Mixed mode chromatography is followed by low pH treatment;
(c) Anion Exchange III is followed by Gel filtration chromatography; and
(d) Gel filtration chromatography is followed by Nano filtration.

3. The process of claim 2, wherein the low pH treatment is at pH 3.7 for 1 hour.

4. The process of claim 1 wherein buffer exchange occurs at least three stages:
(a) After Anion Exchange Chromatography I
(b) After Anion Exchange Chromatography II;
(c) After Affinity Chromatography.

5. The process of claim 1, wherein the process results in percentage purity of about 98.0 to 99.8.

6. The process of claim 1, wherein the recombinant protein is erythropoietin and its analogue darbepoetin alfa.

7. The process of claim 1, wherein the recombinant protein is darbepoetin alfa.
